(19) United States
(12) Patent Application Publication
(10) Pub. No.: US 2009/0209589 A1
(43) Pub. Date: Aug. 20, 2009

(54) N-[(PYRIDIN-2-YL) METHOXY] HETROCYCLYL CARBOXAMIDE DERIVATIVES AND RELATED COMPOUNDS AS FUNGICIDES

(75) Inventors: Pierre-Yves Coqueron, Lyon (FR); Philippe Desbordes, Lyon (FR); Rüdiger Fischer, Pulheim (DE); Oliver Gaertzen, Köln (DE); Pierre Genix, Lyon (FR); Marie-Claire Grosjean-Cournoyer, Curis au Mont d' Or (FR); Benoit Hartmann, Sainte Foy-les-Lyon (FR); Klaus Kunz, Düsseldorf (DE); Darren Mansfield, Bergisch Gladbach (DE); Amos Mattes, Langenfeld (DE); Oswald Ort, Leverkusen (DE); Alain Villier, Collonges au Mont d'Or (FR)

Correspondence Address: Baker Donelson Bearman, Caldwell & Berkowitz, PC, 555 Eleventh Street, NW, Sixth Floor, Washington, DC, 20004, US

(73) Assignee: Bayer Cropscience SA, Lyon (FR)

(21) Appl. No.: 12/307,563

(22) PCT Filed: Jul. 5, 2007
(86) PCT No.: PCT/EP2007/056800

§ 371 Date: Jan. 5, 2009

(30) Foreign Application Priority Data
Jul. 6, 2006 (EP) 06356086.6

(51) Int. Cl. A01N 43/40; C07D 401/12; C07D 409/12; A01P 3/00

(52) U.S. Cl. 514/332; 546/262; 514/343; 546/279.1; 546/280.4; 514/336

(57) ABSTRACT

A compound of general formula (I):

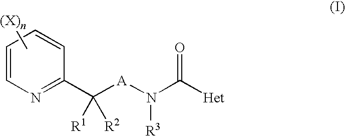

A process for preparing this compound.
A fungicidal composition comprising a compound of general formula (I).
A method for treating plants by applying a compound of general formula (I) or a composition comprising it.

The present invention relates to novel pyridin-2-ylmethyl derivatives, their process of preparation, their use as fungicides, particularly in the form of fungicidal compositions, and methods for the control of phytopathogenic fungi of plants using these compounds or their compositions.

International Patent Application WO 01/11965 describes a broad family of fungicidal compounds of general formula encompassing the compounds of the present invention. However, the said compounds are not described in that patent application and their activity as a fungicide was not tested.

International Patent application WO 03/6456 discloses a broad family of fungicidal compounds. Pyridin-2-ylmethyl derivatives according to the present invention are not disclosed in that patent application.

It is always of high-interest in the field of agrochemicals to use pesticidal compounds more active than the compounds already known by the man ordinary skilled in the art whereby less compound can be used whilst retaining equivalent efficacy.

Furthermore, the provision of new pesticidal compounds with a higher efficacy strongly reduces the risk of appearance of resistant strains in the fungi to be treated.

We have now found a new family of compounds which show enhanced fungicidal activity over the general known family of such compounds.

Accordingly, the present invention relates to a pyridin-2-ylmethyl derivative of general formula (I)

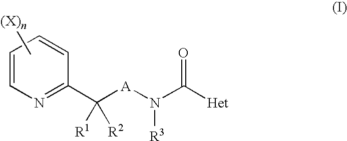

in which:
n is 1, 2, 3 or 4;
X is a halogen atom, a nitro group, a cyano group, a hydroxy group, an amino group, a sulfanyl group, a pentafluoro-λ⁶-sulfanyl group, a formyl group, a formyloxy group, a formylamino group, a carboxy group, a carbamoyl group, a N-hydroxycarbamoyl group, a carbamate group, a (hydroxyimino)-C₁-C₆-alkyl group, a C₁-C₈-alkyl, a C₂-C₈-alkenyl, a C₂-C₈-alkynyl, a tri(C₁-C₈-alkyl)silyl, a C₁-C₈-alkylamino, a di-C₁-C₈-alkylamino, a C₁-C₈-alkoxy, a C₁-C₆-haloalkyl group, a C₁-C₈-halogenoalkoxy having 1 to 5 halogen atoms, a C₁-C₈-alkylsulfanyl, a C₁-C₈-halogenoalkylsulfanyl having 1 to 5 halogen atoms, a C₂-C₈-alkenyloxy, a C₂-C₈-halogenoalkenyloxy having 1 to 5 halogen atoms, a C₃-C₈-alkynyloxy, a C₃-C₈-halogenoalkynyloxy having 1 to 5 halogen atoms, a C₃-C₈-cycloalkyl, a C₃-C₈-halogenocycloalkyl having 1 to 5 halogen atoms, a C₁-C₈-alkylcarbonyl, a C₁-C₈-halogenoalkylcarbonyl having 1 to 5 halogen atoms, a C₁-C₈-alkylcarbamoyl, a di-C₁-C₈-alkylcarbamoyl, a N—C₁-C₈-alkyloxycarbamoyl, a C₁-C₈-alkoxycarbamoyl, a N—C₁-C₈-alkyl-C₁-C₈-alkoxycarbamoyl, a C₁-C₈-alkoxycarbonyl, a C₁-C₈-halogenoalkoxycarbonyl having 1 to 5 halogen atoms, a C₁-C₈-alkylcarbonyloxy, a C₁-C₈-halogenoalkylcarbonyloxy having 1 to 5 halogen atoms, a C₁-C₈-alkylcarbonylamino, a C₁-C₈-halogenoalkylcarbonylamino having 1 to 5 halogen atoms, a C₁-C₈-alkylaminocarbonyloxy, a di-C₁-C₈-alkylaminocarbonyloxy, a C₁-C₈-alkyloxycarbonyloxy, a C₁-C₈-alkylsulphenyl, a C₁-C₈-halogenoalkylsulphenyl having 1 to 5 halogen atoms, a C₁-C₈-alkylsulphinyl, a C₁-C₈-halogenoalkylsulphinyl having 1 to 5 halogen atoms, a C₁-C₈-alkylsulphonyl, a C₁-C₈-halogenoalkylsulphonyl having 1 to 5 halogen atoms, a C₁-C₆-alkoxyimino, a (C₁-C₆-alkoxyimino)-C₁-C₆-alkyl, a (C₁-C₆-alkenyloxyimino)-C₁-C₆-alkyl, a (C₁-C₆-alkynyloxyimino)-C₁-C₆-alkyl, a (benzyloxyimino)-C₁-C₆-alkyl, a benzyloxy, a benzylsulfanyl, a benzylamino, a phenoxy, a phenylsulfanyl or a phenylamino;
A is an oxygen atom, a NR³ group, a sulphur atom, a sulphinyl group, a sulphonyl group or a SiR⁴R⁵ group;
R¹ and R² are chosen independently of each other as being a hydrogen atom, a halogen atom, a cyano group, a hydroxy group, an amino group, a sulfanyl group, a formyl group, a formyloxy group, a formylamino group, a carboxy group, a carbamoyl group, a N-hydroxycarbamoyl group, a carbamate group, a (hydroxyimino)-C₁-C₆-alkyl group, a C₁-C₆-alkyl, a C₂-C₆-alkenyl, a C₂-C₆-alkynyl, a tri(C₁-C₈-alkyl)silyl, a C₁-C₆-alkylamino, a di-C₁-C₆-alkylamino, a C₁-C₆-alkoxy, a C₁-C₆-halogenoalkyl having 1 to 5 halogen atoms, a C₁-C₆-halogenoalkoxy having 1 to 5 halogen atoms, a C₁-C₆-alkylsulfanyl, a C₁-C₆-halogenoalkylsulfanyl having 1 to 5 halogen atoms, a C₂-C₆-alkenyloxy, a C₂-C₆-halogenoalkenyloxy having 1 to 5 halogen atoms, a C₃-C₆-alkynyloxy, a C₃-C₆-halogenoalkynyloxy having 1 to 5 halogen atoms, a C₃-C₆-cycloalkyl, a C₃-C₆-halogenocycloalkyl having 1 to 5 halogen atoms, a C₁-C₆-alkylcarbonyl, a C₁-C₆-halogenoalkylcarbonyl having 1 to 5 halogen atoms, a C₁-C₆-alkylcarbamoyl, a di-C₁-C₆-alkylcarbamoyl, a N—C₁-C₆-alkyloxycarbamoyl, a C₁-C₆-alkoxycarbamoyl, a N—C₁-C₆-alkyl-C₁-C₆-alkoxycarbamoyl, a C₁-C₆-alkoxycarbonyl, a C₁-C₆-halogenoalkoxycarbonyl having 1 to 5 halogen atoms, a C₁-C₆-alkylcarbonyloxy, a C₁-C₆-halogenoalkylcarbonyloxy having 1 to 5 halogen atoms, a C₁-C₆-alkylcarbonylamino, a C₁-C₆-halogenoalkylcarbonylamino having 1 to 5 halogen atoms, a C₁-C₆-alkylaminocarbonyloxy, a di-C₁-C₆-alkylaminocarbonyloxy, a C₁-C₆-alkyloxycarbonyloxy, a C₁-C₆-alkylsulphenyl, a C₁-C₆-halogenoalkylsulphenyl having 1 to 5 halogen atoms, a C₁-C₆-alkylsulphinyl, a C₁-C₆-halogenoalkylsulphinyl having 1 to 5 halogen atoms, a C₁-C₆-alkylsulphonyl, a C₁-C₆-halogenoalkylsulphonyl having 1 to 5 halogen atoms, a benzyl, a benzyloxy, a benzylsulfanyl, a benzylsulfinyl, a benzylsulfonyl, a benzylamino, a phenoxy, a phenylsulfanyl, a phenylsulfinyl, a phenylsulfonyl, a phenylamino, a phenylcarbonylamino or a phenyl group;

with the proviso that R¹ and R² are not both a hydroxyl group;
R³ is a hydrogen atom, a C₁-C₆-alkyl, or a C₃-C₇-cycloalkyl;
R⁴ and R⁵ are chosen independently of each other as being a hydrogen atom, a cyano group, a hydroxy group, a formyl group, a C₁-C₆-alkyl, a C₂-C₆-alkenyl, a C₂-C₆-alkynyl, a tri(C₁-C₈-alkyl)silyl, a C₁-C₆-alkylamino, a di-C₁-C₆-alkylamino, a C₁-C₆-alkoxy, a C₁-C₆-halogenoalkyl having 1 to 5 halogen atoms, a C₁-C₆-halogenoalkoxy having 1 to 5 halogen atoms, a C₂-C₆-alkenyloxy, a C₂-C₆-halogenoalkenyloxy having 1 to 5 halogen atoms, a C₃-C₆-alkynyloxy, a C₃-C₆-halogenoalkynyloxy having 1 to 5 halogen atoms, a C₃-C₆-cycloalkyl, a C₃-C₆-halogenocycloalkyl having 1 to 5 halogen atoms, a C₁-C₆-alkylcarbonyl, a C₁-C₆-halogenoalkylcarbonyl having 1 to 5 halogen atoms, a C₁-C₆-alkylcarbamoyl, a di-C₁-C₆-alkylcarbamoyl, a N—C₁-C₆-alkyloxycarbamoyl, a C₁-C₆-alkoxycarbamoyl, a N—C₁-C₆-alkyl-C₁-C₆-alkoxycarbamoyl, a C₁-C₆-alkoxycarbonyl, a C₁-C₆-halogenoalkoxycarbonyl having 1 to 5 halogen atoms a C₁-C₆-alkylaminocarbonyloxy, a di-C₁-C₆-alkylaminocarbonyloxy, a C₁-C₆-alkyloxycarbonyloxy, a C₁-C₆-alkylsulphenyl, a C₁-C₆-halogenoalkylsulphenyl having 1 to 5 halogen atoms, a C₁-C₆-alkylsulphinyl, a C₁-C₆-halogenoalkylsulphinyl having 1 to 5 halogen atoms, a C₁-C₆-alkylsulphonyl, a C₁-C₆-halogenoalkylsulphonyl having 1 to 5 halogen atoms, a benzyl, a benzylsulfanyl, a benzylsulfinyl, a benzylsulfonyl, a phenylsulfinyl, a phenylsulfonyl, a phenylamino or a phenyl group; and
Het represents a 5-, 6- or 7-membered non-fused heterocycle with one, two or three heteroatoms which may be the same or different, Het being linked by a carbon atom and Het being optionally substituted by one or further substituents chosen independently of each other from a halogen atom, a nitro group, a cyano group, a hydroxy group, an amino group, a sulfanyl group, a pentafluoro-λ⁶-sulfanyl group, a formyl group, a formyloxy group, a formylamino group, a carboxy group, a C₁-C₈-alkyl, a C₁-C₈-halogenoalkyl having 1 to 5 halogen atoms, a C₂-C₈-alkenyl, a C₂-C₈-alkynyl, a C₁-C₈-alkylamino, a di-C₁-C₈-alkylamino, a C₁-C₈-alkoxy, a C₁-C₈-halogenoalkoxy having 1 to 5 halogen atoms, a C₁-C₈-alkoxy-C₂-C₈-alkenyl, a C₁-C₈-alkylsulfanyl, a C₁-C₈-halogenoalkylsulfanyl having 1 to 5 halogen atoms, a C₁-C₈-alkoxycarbonyl, a C₁-C₈-halogenoalkoxycarbonyl having 1 to 5 halogen atoms, a C₁-C₈-alkylcarbonyloxy, a C₁-C₈-halogenoalkylcarbonyloxy having 1 to 5 halogen atoms, a C₁-C₈-alkylsulphenyl, a C₁-C₈-halogenoalkylsulphenyl having 1 to 5 halogen atoms, a C₁-C₈-alkylsulphinyl, a C₁-C₈-halogenoalkylsulphinyl having 1 to 5 halogen atoms, a C₁-C₈-alkylsulphonyl and a C₁-C₈-halogenoalkylsulphonyl having 1 to 5 halogen atoms or a C₁-C₈-alkylsulfonamide;

as well as its salts, N-oxydes, metallic complexes, metalloidic complexes and optically active isomers;

In the context of the present invention:

halogen means fluorine, bromine, chlorine or iodine.

carboxy means —C(═O)OH;

carbonyl means —C(═O)—;

carbamoyl means —C(═O)NH₂;

N-hydroxycarbamoyl means —C(═O)NHOH;

an alkyl group, an alkenyl group, and an alkynyl group as well as moieties containing these terms, can be linear or branched; and

heteroatom means sulphur, nitrogen or oxygen.

In the context of the present invention, it has also to be understood that in the case of di-substituted amino and of di-substituted carbamoyl radicals, the two substituents may form together with the nitrogen atom bearing them a saturated heterocyclic ring containing 3 to 7 atoms.

Any of the compounds of the present invention can exist in one or more optical or chiral isomer forms depending on the number of asymmetric centres in the compound. The invention thus relates equally to all the optical isomers and to their racemic or scalemic mixtures (the term “scalemic” denotes a mixture of enantiomers in different proportions), and to the mixtures of all the possible stereoisomers, in all proportions. The diastereoisomers and/or the optical isomers can be separated according to the methods which are known per se by the man ordinary skilled in the art.

Any of the compounds of the present invention can also exist in one or more geometric isomer forms depending on the number of double bonds in the compound. The invention thus relates equally to all geometric isomers and to all possible mixtures, in all proportions. The geometric isomers can be separated according to general methods, which are known per se by the man ordinary skilled in the art.

Any of the compounds of general formula (I) wherein X represents a hydroxy, a sulfanyl group or an amino group may be found in its tautomeric form resulting from the shift of the proton of said hydroxy, sulfanyl or amino group. Such tautomeric forms of such compounds are also part of the present invention. More generally speaking, all tautomeric forms of compounds of general formula (I) wherein X represents a hydroxy, a sulfanyl group or an amino group, as well as the tautomeric forms of the compounds which can optionally be used as intermediates in the preparation processes, and which will be defined in the description of these processes, are also part of the present invention.

According to the present invention, the 2-pyridyl moiety of compound of general formula (I) may be substituted in any position by (X)ₙ, X and n being as defined above. Preferably, the present invention relates to pyridin-2-ylmethyl derivative of general formula (I) in which the different characteristics may be chosen alone or in combination as being:

as regards n, n is 1 or 2; and

as regards X, X is chosen as being a halogen atom, a cyano group, a (hydroxyimino)-C₁-C₆-alkyl group, a C₁-C₈-alkyl, a C₂-C₈-alkenyl, a C₂-C₈-alkynyl, a tri(C₁-C₈-alkyl)silyl, a C₁-C₈-alkoxy, a C₁-C₆-haloalkyl group, a C₁-C₈-halogenoalkoxy having 1 to 5 halogen atoms, a C₃-C₈-cycloalkyl or a C₃-C₈-halogenocycloalkyl having 1 to 5 halogen atoms.

According to the present invention, the carbon atom of the methylene moiety of compound of formula (I) is substituted by R¹ and R²; R¹ and R² being as defined above. Preferably, the present invention also relates to pyridin-2-ylmethyl derivative of general formula (I) in which R¹ and R² are chosen independently of each other as being a hydrogen atom, a halogen atom, a C₁-C₈-alkyl, a C₂-C₈-alkenyl, a C₂-C₈-alkynyl, a tri(C₁-C₈-alkyl)silyl, a C₁-C₈-alkoxy, a C₁-C₆-haloalkyl group, a C₁-C₈-halogenoalkoxy having 1 to 5 halogen atoms, a C₃-C₈-cycloalkyl or a C₃-C₈-halogenocycloalkyl having 1 to 5 halogen atoms.

According to the present invention, the “A” atom of compound of formula (I) is chosen as being an oxygen atom, a NR³ group, a sulphur atom, a sulphinyl group, a sulphonyl group or a SiR⁴R⁵ group; R⁴ and R⁵ being as defined above. Preferably, the present invention also relates to pyridin-2-ylmethyl derivative of general formula (I) in which the different characteristics may be chosen alone or in combination as being:

A is chosen as being an oxygen atom, a nitrogen atom, a sulphur atom; and

R⁴ and R⁵ are chosen independently of each other as being a hydrogen atom, a C₁-C₈-alkyl, a C₂-C₈-alkenyl, a C₂-C₈-alkynyl, a C₁-C₈-alkoxy, a C₁-C₆-haloalkyl group, a C₁-C₈-halogenoalkoxy having 1 to 5 halogen atoms, a C₃-C₈-cycloalkyl or a C₃-C₈-halogenocycloalkyl having 1 to 5 halogen atoms.

According to the present invention, the nitrogen atom of the carboxamide moiety of the compound of formula (I) is substituted by R⁵, R⁵ being a hydrogen atom, a C₁-C₆-alkyl or a C₃-C₇-cycloalkyl. Preferably, the C₃-C₇-cycloalkyl is cyclopropyl.

According to the present invention, “Het” of the compound of general formula (I) is a 5-, 6- or 7-membered non-fused heterocycle with one, two or three heteroatoms which may be the same or different, Het being linked by a carbon atom and being optionally substituted. Preferably, the present invention also relates to pyridin-2-ylmethyl derivative of general formula (I) in which the different characteristics may be chosen alone or in combination as being:

Het is chosen as being 2-furan, 3-furan, 4,5-dihydro-3-furan, 2-thiophene, 3-thiophene, 2-pyrrole, 3-pyrrole, 5-oxazole, 4-oxazole, 5-thiazole, 4-thiazole, 5-pyrazole, 4-pyrazole, 3-pyrazole, 3-isoxazole, 4-isoxazole, 5-isoxazole, 3-isothiazole, 4-1,2,3-triazole, 4-thiadiazole, 5-thiadiazole, 2-pyridine, 3-pyridine, 4-pyridine, 2-oxathiine, 4,5-dihydro-3-pyran, 4,5-dihydro-2-thiopyran, 4,5-dihydro-3-thiopyran or 2-pyrazine;

Het is substituted in ortho position;

Het is substituted by a halogen atom, a C₁-C₆-alkyl, an amino group, a C₁-C₈-alkylamino or a di-C₁-C₈-alkylamino.

According to the present invention, “Het” of the compound of general formula (I) may be a five membered ring heterocycle. Specific examples of compounds of the present invention where Het is a five membered heterocycle include:

Het represents a heterocycle of the general formula (Het-1)

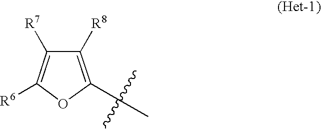

in which:
R⁶ and R⁷ may be the same or different and may be a hydrogen atom, a halogen atom, an amino group, a nitro group, a C₁-C₄-alkyl or a C₁-C₄-halogenoalkyl having 1 to 5 halogen atoms; and
R⁸ may be a halogen atom, a nitro group, a C₁-C₄-alkyl or a C₁-C₄-halogenoalkyl having 1 to 5 halogen atoms.

Het represents a heterocycle of the general formula (Het-2)

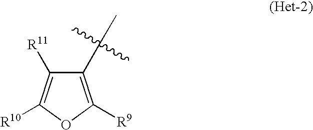

in which:
R⁹ may be a hydrogen atom, a halogen atom, a C₁-C₄-alkyl or a C₁-C₄-halogenoalkyl having 1 to 5 halogen atoms; and

R¹⁰ and R¹¹ may be the same or different and may be a hydrogen atom, a halogen atom, an amino group, a C₁-C₄-alkyl or a C₁-C₄-halogenoalkyl having 1 to 5 halogen atoms;

provided that the R⁹ and R¹¹ are not both a hydrogen atom.

Het represents a heterocycle of the general formula (Het-3)

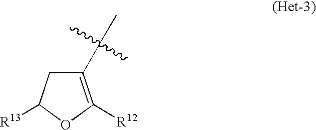

in which:
R¹² may be a halogen atom, a C₁-C₄-alkyl or a C₁-C₄-halogenoalkyl having 1 to 5 halogen atoms; and
R¹³ may be a hydrogen atom, a C₁-C₄-alkyl or a C₁-C₄-halogenoalkyl having 1 to 5 halogen atoms.

Het represents a heterocycle of the general formula (Het-4)

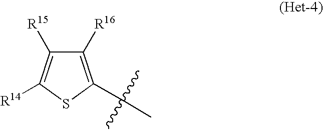

in which:
R¹⁴ and R¹⁵ may be the same or different and may be a hydrogen atom, a halogen atom, a C₁-C₄-alkyl, a C₁-C₄-halogenoalkyl having 1 to 5 halogen atoms, a C₁-C₄-alkylthio, a C₁-C₄-alkylsulphonyl, a phenyl optionally substituted by a halogen atom or a C₁-C₄-alkyl or a pyridyl optionally substituted by a halogen atom or a C₁-C₄-alkyl; and
R¹⁶ may be a halogen atom, a cyano group, a C₁-C₄-alkyl, a C₁-C₄-halogenoalkyl having 1 to 5 halogen atoms or a C₁-C₄-halogenoalkoxy having 1 to 5 halogen atoms.

Het represents a heterocycle of the general formula (Het-5)

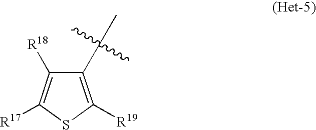

in which:
R¹⁷ and R¹⁸ may be the same or different and may be a hydrogen atom, a halogen atom, a C₁-C₄-alkyl, a C₁-C₄-alkyloxy or a C₁-C₄-halogenoalkyl having 1 to 5 halogen atoms; and
R¹⁹ may be a hydrogen atom, a halogen atom, a C₁-C₄-alkyl or a C₁-C₄-halogenoalkyl having 1 to 5 halogen atoms;

provided that the R¹⁸ and R¹⁹ are not both a hydrogen atom.

Het represents a heterocycle of the general formula (Het-6)

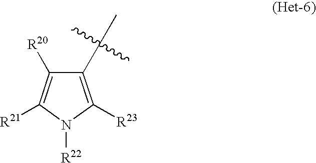

in which:
R²⁰ may be a hydrogen atom, a halogen atom, a cyano group, a C₁-C₄-alkyl or a C₁-C₄-halogenoalkyl having 1 to 5 halogen atoms;
R²¹ and R²³ may be the same or different and may be a hydrogen atom, a halogen atom, a C₁-C₄-alkyl or a C₁-C₄-halogenoalkyl having 1 to 5 halogen atoms; and
R²² may be a hydrogen atom, a cyano group, a C₁-C₄-alkyl, a C₁-C₄-halogenoalkyl having 1 to 5 halogen atoms, a C₁-C₄-alkoxy-C₁-C₄-alkyl, a hydroxy-C₁-C₄-alkyl, a C₁-C₄-alkylsulphonyl, a di(C₁-C₄-alkyl)aminosulphonyl, a C₁-C₆-alkylcarbonyl, a phenylsulphonyl optionally substituted by a halogen atom or a C₁-C₄-alkyl, or a benzoyl optionally substituted by a halogen atom or a C₁-C₄-alkyl;

provided that the R²⁰ and R²³ are not both a hydrogen atom.

Het represents a heterocycle of the general formula (Het-7)

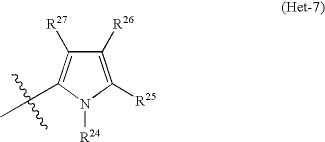

in which:
R²⁴ may be a hydrogen atom, a cyano group, a C₁-C₄-alkyl, a C₁-C₄-halogenoalkyl having 1 to 5 halogen atoms, a C₁-C₄-alkoxy-C₁-C₄-alkyl, a hydroxy-C₁-C₄-alkyl, a C₁-C₄-alkylsulphonyl, a di(C₁-C₄-alkyl)aminosulphonyl, a C₁-C₆-alkylcarbonyl, a phenylsulphonyl optionally substituted by a halogen atom or a C₁-C₄-alkyl, or a benzoyl optionally substituted by a halogen atom or a C₁-C₄-alkyl; and
R²⁵, R²⁶ and R²⁷ may be the same or different and may be a hydrogen atom, a halogen atom, a cyano group, a C₁-C₄-alkyl, a C₁-C₄-halogenoalkyl having 1 to 5 halogen atoms or a C₁-C₄-alkylcarbonyl;

provided that R²⁴ and R²⁷ are not both a hydrogen atom.

Het represents a heterocycle of the general formula (Het-8)

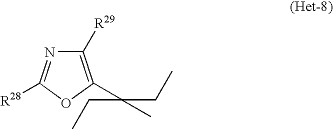

in which:
R²⁸ may be a hydrogen atom or a C₁-C₄-alkyl; and
R²⁹ may be a halogen atom, a C₁-C₄-alkyl or a C₁-C₄-halogenoalkyl having 1 to 5 halogen atoms.

Het represents a heterocycle of the general formula (Het-9)

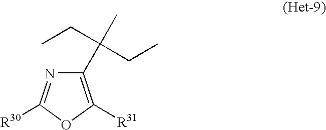

in which:
R³⁰ may be a hydrogen atom or a C₁-C₄-alkyl; and
R³¹ may be a halogen atom, a C₁-C₄-alkyl, a C₁-C₄-halogenoalkyl having 1 to 5 halogen atoms or a phenyl optionally substituted by a halogen atom or a C₁-C₄-alkyl.

Het represents a heterocycle of the general formula (Het-10)

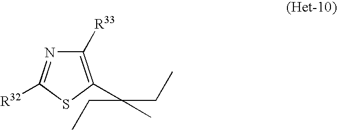

in which:
R³² may be a hydrogen atom, a halogen atom, an amino group, a cyano group, a C₁-C₄-alkylamino, a di-(C₁-C₄-alkyl)amino, a C₁-C₄-alkyl, a C₁-C₄-halogenoalkyl having 1 to 5 halogen atoms or a phenyl optionally substituted by a halogen atom or a C₁-C₄-alkyl; and
R³³ may be a halogen atom, a C₁-C₄-alkyl or a C₁-C₄-halogenoalkyl having 1 to 5 halogen atoms.

Het represents a heterocycle of the general formula (Het-11)

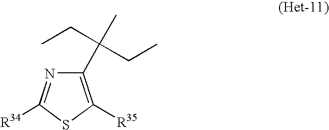

in which:
R³⁴ may be a hydrogen atom, a halogen atom, an amino group, a cyano group, a C₁-C₄-alkylamino, a di-(C₁-C₄-alkyl)amino, a C₁-C₄-alkyl or a C₁-C₄-halogenoalkyl having 1 to 5 halogen atoms; and
R³⁵ may be a halogen atom, a C₁-C₄-alkyl or a C₁-C₄-halogenoalkyl having 1 to 5 halogen atoms.

Het represents a heterocycle of the general formula (Het-12)

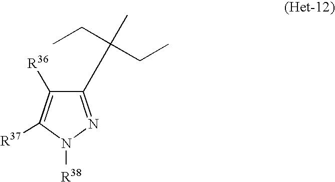

in which:
R³⁶ may be a halogen atom, a cyano group, a nitro group, a C₁-C₄-alkyl, a C₁-C₄-halogenoalkyl having 1 to 5 halogen atoms, a C₃-C₆-cycloalkyl, a C₁-C₄-alkoxy, a C₁-C₄-halogenoalkoxy having 1 to 5 halogen atoms, a C₁-C₄-alkylthio, a C₁-C₄-halogenoalkylthio having 1 to 5 halogen atoms, an aminocarbonyl group or an aminocarbonyl-C₁-C₄-alkyl;
R³⁷ may be a hydrogen atom, a halogen atom, a cyano group, a nitro group, a C₁-C₄-alkyl, a C₁-C₄-alkoxy or a C₁-C₄-alkylthio; and
R³⁸ may be a hydrogen atom, a phenyl, a C₁-C₄-alkyl, a C₁-C₄-halogenoalkyl having 1 to 5 halogen atoms, a hydroxy-C₁-C₄-alkyl, a C₂-C₆-alkenyl, a C₃-C₆-cycloalkyl, a C₁-C₄-alkylthio-C₁-C₄-alkyl, a C₁-C₄-halogenoalkylthio-C₁-C₄-alkyl having 1 to 5 halogen atoms, a C₁-C₄-alkoxy-C₁-C₄-alkyl or a C₁-C₄-halogenoalkoxy-C₁-C₄-alkyl having 1 to 5 halogen atoms.

Het represents a heterocycle of the general formula (Het-13)

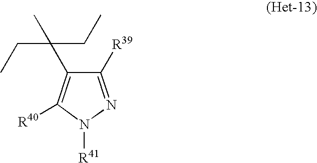

in which:
R³⁹ may be a hydrogen atom, a halogen atom, a cyano group, a nitro group, a C₁-C₄-alkyl, a C₁-C₄-halogenoalkyl having 1 to 5 halogen atoms, a C₃-C₆-cycloalkyl, a C₁-C₄-alkoxy, a C₁-C₄-halogenoalkoxy having 1 to 5 halogen atoms, a C₁-C₄-alkylthio, a C₁-C₄-halogenoalkylthio having 1 to 5 halogen atoms, an aminocarbonyl or an aminocarbonyl-C₁-C₄-alkyl;
R⁴⁰ may be a hydrogen atom, a halogen atom, a cyano group, a C₁-C₄-alkyl, a C₁-C₄-alkoxy, a C₁-C₄-halogenoalkoxy having 1 to 5 halogen atoms or a C₁-C₄-alkylthio; and
R⁴¹ may be a hydrogen atom, a C₁-C₄-alkyl, a C₁-C₄-halogenoalkyl having 1 to 5 halogen atoms, a hydroxy-C₁-C₄-alkyl, a C₂-C₆-alkenyl, a C₃-C₆-cycloalkyl, a C₁-C₄-alkylthio-C₁-C₄-alkyl, a C₁-C₄-halogenoalkylthio-C₁-C₄-alkyl having 1 to 5 halogen atoms, a C₁-C₄-alkoxy-C₁-C₄-alkyl, a C₁-C₄-halogenoalkoxy-C₁-C₄-alkyl having 1 to 5 halogen atoms or a phenyl optionally substituted by a halogen atom, a C₁-C₄-alkyl, a C₁-C₄-alkoxyalkyl or a nitro group;

provided that the R³⁹ and R⁴⁰ are not both a hydrogen atom.

Het represents a heterocycle of the general formula (Het-14)

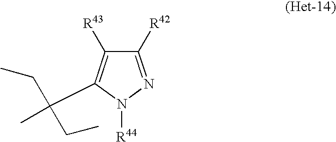

in which:
R⁴² may be a hydrogen atom, a halogen atom, a cyano group, a nitro group, a C₁-C₄-alkyl, a C₁-C₄-halogenoalkyl having 1 to 5 halogen atoms, a C₃-C₆-cycloalkyl, a C₁-C₄-alkoxy, a C₁-C₄-halogenoalkoxy having 1 to 5 halogen atoms, a C₁-C₄-alkylthio, a C₁-C₄-halogenoalkylthio having 1 to 5 halogen atoms, an aminocarbonyl, or an aminocarbonyl-C₁-C₄-alkyl;
R⁴³ may be a hydrogen atom, a halogen atom, a cyano group, a C₁-C₄-alkyl, a C₁-C₄-alkoxy, a C₁-C₄-alkylthio or a C₁-C₄-halogenoalky having 1 to 5 halogen atoms;
R⁴⁴ may be a hydrogen atom, a phenyl, a benzyl, a C₁-C₄-alkyl, a C₁-C₄-halogenoalkyl having 1 to 5 halogen atoms, a hydroxy-C₁-C₄-alkyl, a C₂-C₆-alkenyl, a C₃-C₆-cycloalkyl, a C₁-C₄-alkylthio-C₁-C₄-alkyl, a C₁-C₄-halogenoalkylthio-C₁-C₄-alkyl having 1 to 5 halogen atoms, a C₁-C₄-alkoxy-C₁-C₄-alkyl, a C₁-C₄-halogenoalkoxy-C₁-C₄-alkyl having 1 to 5 halogen atoms;

provided that R⁴³ and R⁴⁴ are not both a hydrogen atom.

Het represents a heterocycle of the general formula (Het-15)

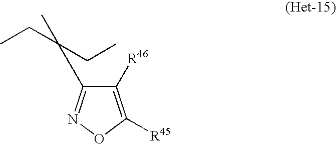

in which:
R⁴⁵ may be a hydrogen atom, a halogen atom, a C₁-C₄-alkyl or a C₁-C₄-halogenoalkyl having 1 to 5 halogen atoms; and
R⁴⁶ may be a halogen atom, a C₁-C₄-alkyl or a C₁-C₄-halogenoalkyl having 1 to 5 halogen atoms.

Het represents a heterocycle of the general formula (Het-16)

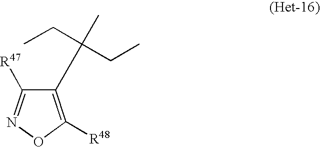

in which R⁴⁷ and R⁴⁸ may be the same or different and may be a hydrogen atom, a halogen atom, a C₁-C₄-alkyl, a C₁-C₄-halogenoalkyl having 1 to 5 halogen atoms, a phenyl optionally substituted by a halogen atom or a C₁-C₄-alkyl, or a heterocyclyl optionally substituted by a halogen atom or a C₁-C₄-alkyl;

provided that R⁴⁷ and R⁴⁸ are not both a hydrogen atom.

Het represents a heterocycle of the general formula (Het-17)

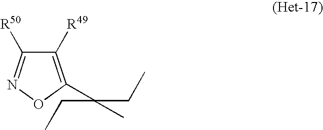

in which
R⁴⁹ may be a halogen atom, a C₁-C₄-alkyl or a C₁-C₄-halogenoalkyl having 1 to 5 halogen atoms. and
R⁵⁰ may be a halogen atom, a C₁-C₄-alkyl or a C₁-C₄-halogenoalkyl having 1 to 5 halogen atoms.

Het represents a heterocycle of the general formula (Het-18)

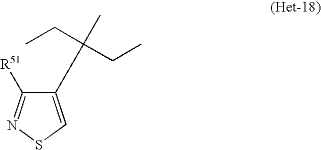

in which R⁵¹ may be a halogen atom, a C₁-C₄-alkyl or a C₁-C₄-halogenoalkyl having 1 to 5 halogen atoms.

Het represents a heterocycle of the general formula (Het-19)

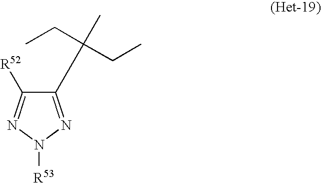

in which:
R⁵² may be a halogen atom, a C₁-C₄-alkyl or a C₁-C₄-halogenoalkyl having 1 to 5 halogen atoms; and
R⁵³ may be a hydrogen atom, a C₁-C₄-alkyl, a C₁-C₄-halogenoalkyl having 1 to 5 halogen atoms, or a phenyl optionally substituted by a halogen atom or a C₁-C₄-alkyl.

Het represents a heterocycle of the general formula (Het-20)

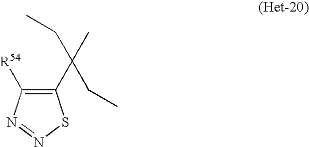

in which R⁵⁴ may be a halogen atom, a C₁-C₄-alkyl or a C₁-C₄-halogenoalkyl having 1 to 5 halogen atoms.

According to the present invention, “Het” of the compound of general formula (I) may be a six membered ring heterocycle. Specific examples of compounds of the present invention where Het is a six membered heterocycle include:

Het represents a heterocycle of the general formula (Het-21)

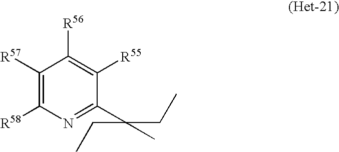

in which:
R⁵⁵ may be a halogen atom, a hydroxy group, a cyano group, a C₁-C₄-alkyl, a C₁-C₄-halogenoalkyl having 1 to 5 halogen atoms, a C₁-C₄-alkoxy, a C₁-C₄-alkylthio, a C₁-C₄-halogenoalkylthio having 1 to 5 halogen atoms or a C₁-C₄-halogenoalkoxy having 1 to 5 halogen atoms;
R⁵⁶, R⁵⁷ and R⁵⁸, which may be the same or different, may be a hydrogen atom, a halogen atom, a cyano group, a C₁-C₄-alkyl, a C₁-C₄-halogenoalkyl having 1 to 5 halogen atoms, a C₁-C₄-alkoxy, a C₁-C₄-alkylthio, a C₁-C₄-halogenoalkoxy having 1 to 5 halogen atoms, a C₁-C₄-alkylsulphinyl or a C₁-C₄-alkylsulphonyl.

Het represents a heterocycle of the general formula (Het-22)

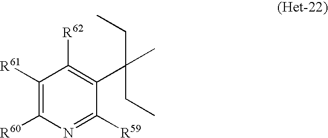

in which:
R⁵⁹ may be a hydrogen atom, a halogen atom, a hydroxy group, a cyano group, a C₁-C₄-alkyl, a C₁-C₄-halogenoalkyl having 1 to 5 halogen atoms, a C₁-C₄-alkoxy, a C₁-C₈-alkylthio, a C₂-C₈-alkenylthio a C₁-C₄-halogenoalkylthio having 1 to 5 halogen atoms, a C₁-C₄-halogenoalkoxy having 1 to 5 halogen atoms, a phenyloxy optionally substituted by a halogen atom or a C₁-C₄-alkyl, or a phenylthio optionally substituted by a halogen atom or a C₁-C₄-alkyl;
R⁶⁰, R⁶¹ and R⁶², which may the same or different, may be a hydrogen atom, a halogen atom, a cyano group, a C₁-C₄-alkyl, a C₁-C₄-halogenoalkyl having 1 to 5 halogen atoms, a C₁-C₄-alkoxy, a C₁-C₄-alkylthio, a C₁-C₄-halogenoalkoxy having 1 to 5 halogen atoms, a C₁-C₄-alkylsulphinyl, a C₁-C₄-alkylsulphonyl or a N-morpholine optionally substituted by a halogen atom or a C₁-C₄-alkyl, or a thienyl optionally substituted by a halogen atom or a C₁-C₄-alkyl;

provided that the R⁵⁹ and R⁶² are not both a hydrogen atom.

Het represents a heterocycle of the general formula (Het-23)

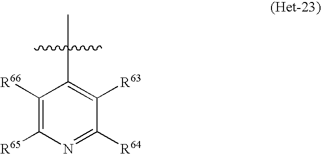

in which R⁶³, R⁶⁴, R⁶⁵ and R⁶⁶, which may be the same or different, may be a hydrogen atom, a halogen atom, a hydroxy group, a cyano group, a C₁-C₄-alkyl, a C₁-C₄-halogenoalkyl having 1 to 5 halogen atoms, a C₁-C₄-alkoxy, a C₁-C₄-alkylthio, a C₁-C₄-halogenoalkylthio having 1 to 5 halogen atoms, a C₁-C₄-halogenoalkoxy having 1 to 5 halogen atoms, a C₁-C₄-alkylsulphinyl or a C₁-C₄-alkylsulphonyl;

provided that the R⁶³ and R⁶⁶ are not both a hydrogen atom.

Het represents a heterocycle of the general formula (Het-24)

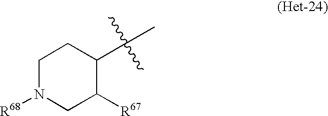

in which:
R⁶⁷ may be a halogen atom, a C₁-C₄-alkyl or a C₁-C₄-halogenoalkyl having 1 to 5 halogen atoms;
R⁶⁸ may be a hydrogen atom, a C₁-C₄-alkyl, a C₁-C₄-halogenoalkyl having 1 to 5 halogen atoms, a C₁-C₆-alkoxycarbonyl, a benzyl optionally substituted by 1 to 3 halogen atoms, a benzyloxycarbonyl optionally substituted by 1 to 3 halogen atoms or a heterocyclyl.

Het represents a heterocycle of the general formula (Het-25)

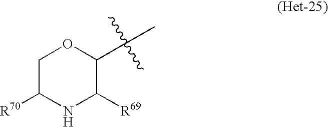

in which:
R⁶⁹ may be a halogen atom, a hydroxy group, a cyano group, a C₁-C₄-alkyl, a C₁-C₄-halogenoalkyl having 1 to 5 halogen atoms, a C₁-C₄-alkoxy, a C₁-C₄-alkylthio, a C₁-C₄-halogenoalkylthio having 1 to 5 halogen atoms or a C₁-C₄-halogenoalkoxy having 1 to 5 halogen atoms;
R⁷⁰ may be a hydrogen atom, a C₁-C₄-alkyl, a C₁-C₄-halogenoalkyl having 1 to 5 halogen atoms or a benzyl.

Het represents a heterocycle of the general formula (Het-26)

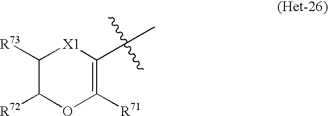

in which:
X¹ may be a sulphur atom, —SO—, —SO₂— or —CH₂—;
R⁷¹ may be a C₁-C₄-alkyl or a C₁-C₄-halogenoalkyl having 1 to 5 halogen atoms; and

R⁷² and R⁷³ may be the same or different and may be a hydrogen atom or a C₁-C₄-alkyl.

Het represents a heterocycle of the general formula (Het-27)

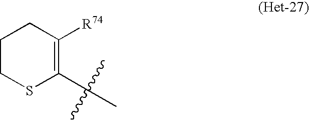

in which:
R⁷⁴ may be a C₁-C₄-alkyl or a C₁-C₄-halogenoalkyl having 1 to 5 halogen atoms;

Het represents a heterocycle of the general formula (Het-28)

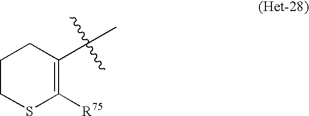

in which:
R⁷⁵ may be a C₁-C₄-alkyl or a C₁-C₄-halogenoalkyl having 1 to 5 halogen atoms.

Het represents a heterocycle of the general formula (Het-29)

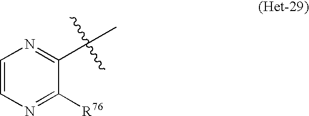

in which R⁷⁶ may be a halogen atom, a C₁-C₄-alkyl or a C₁-C₄-halogenoalkyl having 1 to 5 halogen atoms.

The present invention also relates to a process for the preparation of the compound of general formula (I). Thus, according to a further aspect of the present invention there is provided a process for the preparation of compound of general formula (I) as defined above, which comprises reacting a pyridin-2-ylmethyl derivative of general formula (II) or one of its salt:

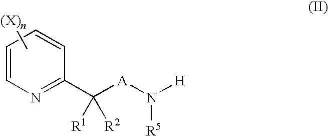

in which X, n, R¹, R², R⁵ and A are as defined above; with a carboxylic acid derivative of the general formula (III)

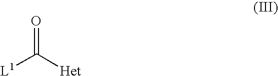

in which:
Y and p are as defined above
L¹ is a leaving group chosen as being a halogen atom, a hydroxyl group, —OR^{a}, —OCOR^{a}, R^{a} being a C₁-C₆ alkyl, a C₁-C₆ haloalkyl, a benzyl, 4-methoxybenzyl, pentafluorophenyl or a group of formula

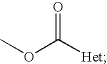

in the presence of a catalyst and, if L¹ is a hydroxyl group, in the presence of a condensing agent.

The process according to the present invention is conducted in the presence of a catalyst. Suitable catalyst may be chosen as being 4-dimethyl-aminopyridine, 1-hydroxy-benzotriazole or dimethylformamide.

In case L¹ is a hydroxy group, the process according to the present invention is conducted in the presence of condensing agent. Suitable condensing agent may be chosen as being acid halide former, such as phosgene, phosphorous tribromide, phosphorous trichloride, phosphorous pentachloride, phosphorous trichloride oxide or thionyl chloride; anhydride former, such as ethyl chloroformate, methyl chloroformate, isopropyl chloroformate, isobutyl chloroformate or methanesulfonylchloride; carbodiimides, such as N,N′-dicyclohexylcarbodiimide (DCC) or other customary condensing agents, such as phosphorous pentoxide, polyphosphoric acid, N,N′-carbonyl-diimidazole, 2-ethoxy-N-ethoxycarbonyl-1,2-dihydroquinoline (EEDQ), triphenylphosphine/tetrachloromethane, 4-(4,6-dimethoxy[1.3.5]triazin-2-yl)-4-methylmorpholinium chloride hydrate or bromo-tripyrrolidino-phosphonium-hexafluorophosphate.

The compound according to the present invention can be prepared according to the general processes of preparation described above. It will nevertheless be understood that, on the basis of his general knowledge and of available publications, the skilled worker will be able to adapt this method according to the specifics of each of the compounds, which it is desired to synthesise.

On the basis of his general knowledge and of available publications, the skilled worker will also be able to prepare intermediate compound of formula (II) according to the present invention.

The present invention also relates to a fungicidal composition comprising an effective amount of an active material of general formula (I). Thus, according to the present invention, there is provided a fungicidal composition comprising, as an active ingredient, an effective amount of a compound of general formula (I) as defined above and an agriculturally acceptable support, carrier or filler.

In the present specification, the term “support” denotes a natural or synthetic, organic or inorganic material with which the active material is combined to make it easier to apply, notably to the parts of the plant. This support is thus generally inert and should be agriculturally acceptable. The support may be a solid or a liquid.

Examples of suitable supports include clays, natural or synthetic silicates, silica, resins, waxes, solid fertilisers, water, alcohols, in particular butanol, organic solvents, mineral and plant oils and derivatives thereof. Mixtures of such supports may also be used.

The composition may also comprise additional components. In particular, the composition may further comprise a surfactant. The surfactant can be an emulsifier, a dispersing agent or a wetting agent of ionic or non-ionic type or a mixture of such surfactants. Mention may be made, for example, of polyacrylic acid salts, lignosulphonic acid salts, phenolsulphonic or naphthalenesulphonic acid salts, polycondensates of ethylene oxide with fatty alcohols or with fatty acids or with fatty amines, substituted phenols (in particular alkylphenols or arylphenols), salts of sulphosuccinic acid esters, taurine derivatives (in particular alkyl taurates), phosphoric esters of polyoxyethylated alcohols or phenols, fatty acid esters of polyols, and derivatives of the above compounds containing sulphate, sulphonate and phosphate functions. The presence of at least one surfactant is generally essential when the active material and/or the inert support are water-insoluble and when the vector agent for the application is water. Preferably, surfactant content may be comprised between 5% and 40% by weight of the composition.

Optionally, additional components may also be included, e.g. protective colloids, adhesives, thickeners, thixotropic agents, penetration agents, stabilisers, sequestering agents. More generally, the active materials can be combined with any solid or liquid additive, which complies with the usual formulation techniques.

In general, the composition according to the invention may contain from 0.05 to 99% (by weight) of active material, preferably 10 to 70% by weight.

Compositions according to the present invention can be used in various forms such as aerosol dispenser, capsule suspension, cold fogging concentrate, dustable powder, emulsifiable concentrate, emulsion oil in water, emulsion water in oil, encapsulated granule, fine granule, flowable concentrate for seed treatment, gas (under pressure), gas generating product, granule, hot fogging concentrate, macrogranule, microgranule, oil dispersible powder, oil miscible flowable concentrate, oil miscible liquid, paste, plant rodlet, powder for dry seed treatment, seed coated with a pesticide, soluble concentrate, soluble powder, solution for seed treatment, suspension concentrate (flowable concentrate), ultra low volume (ulv) liquid, ultra low volume (ulv) suspension, water dispersible granules or tablets, water dispersible powder for slurry treatment, water soluble granules or tablets, water soluble powder for seed treatment and wettable powder.

These compositions include not only compositions which are ready to be applied to the plant or seed to be treated by means of a suitable device, such as a spraying or dusting device, but also concentrated commercial compositions which must be diluted before application to the crop.

The compounds of the invention can also be mixed with one or more insecticides, fungicides, bactericides, attractant acaricides or pheromones or other compounds with biological activity. The mixtures thus obtained have a broadened spectrum of activity. The mixtures with other fungicides are particularly advantageous. Examples of suitable fungicide mixing partners may be selected in the following lists

1) a compound capable to inhibit the nucleic acid synthesis like benalaxyl, benalaxyl-M, bupirimate, clozylacon, dimethirimol, ethirimol, furalaxyl, hymexazol, mefenoxam, metalaxyl, metalaxyl-M, ofurace, oxadixyl, oxolinic acid;

2) a compound capable to inhibit the mitosis and cell division like benomyl, carbendazim, diethofencarb, ethaboxam, fuberidazole, pencycuron, thiabendazole, thiophanate-methyl, zoxamide;

3) a compound capable to inhibit the respiration for example as CI-respiration inhibitor like diflumetorim;

as CII-respiration inhibitor like boscalid, carboxin, fenfuram, flutolanil, furametpyr, furmecyclox, mepronil, oxycarboxin, penthiopyrad, thifluzamide;

as CIII-respiration inhibitor like amisulbrom, azoxystrobin, cyazofamid, dimoxystrobin, enestrobin, famoxadone, fenamidone, fluoxastrobin, kresoxim-methyl, metominostrobin, orysastrobin, picoxystrobin, pyraclostrobin, trifloxystrobin;

4) a compound capable of to act as an uncoupler like dinocap, fluazinam, meptyldinocap;

5) a compound capable to inhibit ATP production like fentin acetate, fentin chloride, fentin hydroxide, silthiofam;

6) a compound capable to inhibit AA and protein biosynthesis like andoprim, blasticidin-S, cyprodinil, kasugamycin, kasugamycin hydrochloride hydrate, mepanipyrim, pyrimethanil;

7) a compound capable to inhibit the signal transduction like fenpiclonil, fludioxonil, quinoxyfen;

8) a compound capable to inhibit lipid and membrane synthesis like biphenyl, chlozolinate, edifenphos, etridiazole, iodocarb, iprobenfos, iprodione, isoprothiolane, procymidone, propamocarb, propamocarb hydrochloride, pyrazophos, tolclofos-methyl, vinclozolin;

9) a compound capable to inhibit ergosterol biosynthesis like aldimorph, azaconazole, bitertanol, bromuconazole, cyproconazole, diclobutrazole, difenoconazole, diniconazole, diniconazole-M, dodemorph, dodemorph acetate, epoxiconazole, etaconazole, fenarimol, fenbuconazole, fenhexamid, fenpropidin, fenpropimorph, fluquinconazole, flurprimidol, flusilazole, flutriafol, furconazole, furconazole-cis, hexaconazole, imazalil, imazalil sulfate, imibenconazole, ipconazole, metconazole, myclobutanil, naftifine, nuarimol, oxpoconazole, paclobutrazol, pefurazoate, penconazole, prochloraz, propiconazole, prothioconazole, pyributicarb, pyrifenox, simeconazole, spiroxamine, tebuconazole, terbinafine, tetraconazole, triadimefon, triadimenol, tridemorph, triflumizole, triforine, triticonazole, uniconazole, viniconazole, voriconazole;

10) a compound capable to inhibit cell wall synthesis like benthiavalicarb, dimethomorph, flumorph, iprovalicarb, mandipropamid, polyoxins, polyoxorim, validamycin A;

11) a compound capable to inhibit melanine biosynthesis like carpropamid, diclocymet, fenoxanil, phthalide, pyroquilon, tricyclazole;

12) a compound capable to induce a host defence like acibenzolar-S-methyl, probenazole, tiadinil;

13) a compound capable to have a multisite action like Bordeaux mixture, captafol, captan, chlorothalonil, copper naphthenate, copper oxide, copper oxychloride, copper preparations such as copper hydroxide, copper sulphate, dichlofluanid, dithianon, dodine, dodine free base, ferbam, fluorofolpet, folpet, guazatine, guazatine acetate, iminoctadine, iminoctadine albesilate, iminoctadine triacetate, mancopper, mancozeb, maneb, metiram, metiram zinc, oxine-copper, propineb, sulphur and sulphur preparations including calcium polysulphide, thiram, tolylfluanid, zineb, ziram;

14) a compound selected in the following list: (2E)-2-(2-{[6-(3-chloro-2-methylphenoxy)-5-fluoropyrimidin-4-yl]oxy}phenyl)-2-(methoxyimino)-N-methylacetamide, (2E)-2-{2-[({[(1 E)-1-(3-{[(E)-1-fluoro-2-phenylvinyl]oxy}phenyl)ethylidene]amino}oxy)methyl]phenyl}-2-(methoxyimino)-N-methylacetamide, 1-(4-chlorophenyl)-2-(1H-1,2,4-triazol-1-yl)cycloheptanol, 1-[(4-methoxyphenoxy)methyl]-2,2-dimethylpropyl-1H-imidazole-1-carboxylate, 1-methyl-N-[2-(1,1,2,2-tetrafluoroethoxy)phenyl]-3-(trifluoromethyl)-1H-pyrazole-4-carboxamide, 2,3,5,6-tetrachloro-4-(methylsulfonyl)pyridine, 2-butoxy-6-iodo-3-propyl-4H-chromen-4-one, 2-chloro-N-(1,1,3-trimethyl-2,3-dihydro-1H-inden-4-yl)nicotinamide, 2-phenylphenol and salts, 3-(difluoromethyl)-1-methyl-N-[2-(1,1,2,2-tetrafluoroethoxy)phenyl]-1H-pyrazole-4-carboxamide, 3-(difluoromethyl)-N-[(9R)-9-isopropyl-1,2,3,4-tetrahydro-1,4-methanonaphthalen-5-yl]-1-methyl-1H-pyrazole-4-carboxamide, 3-(difluoromethyl)-N-[(9S)-9-isopropyl-1,2,3,4-tetrahydro-1,4-methanonaphthalen-5-yl]-1-methyl-1H-pyrazole-4-carboxamide, 3-(difluoromethyl)-N-[4′-(3,3-dimethylbut-1-yn-1-yl)biphenyl-2-yl]-1-methyl-1H-pyrazole-4-carboxamide, 3,4,5-trichloropyridine-2,6-dicarbonitrile, 3-[5-(4-chlorophenyl)-2,3-dimethylisoxazolidin-3-yl]pyridine, 3-chloro-5-(4-chlorophenyl)-4-(2,6-difluorophenyl)-6-methylpyridazine, 4-(4-chlorophenyl)-5-(2,6-difluorophenyl)-3,6-dimethylpyridazine, 5-chloro-7-(4-methylpiperidin-1-yl)-6-(2,4,6-trifluorophenyl)[1,2,4]triazolo[1,5-a]pyrimidine, 8-hydroxyquinoline sulfate, benthiazole, bethoxazin, capsimycin, carvone, chinomethionat, cufraneb, cyflufenamid, cymoxanil, dazomet, debacarb, dichlorophen, diclomezine, dicloran, difenzoquat, difenzoquat methylsulphate, diphenylamine, ecomate, ferimzone, flumetover, fluopicolide, fluoroimide, flusulfamide, fosetyl-aluminium, fosetyl-calcium, fosetyl-sodium, hexachlorobenzene, irumamycin, isotianil, methasulfocarb, methyl (2E)-2-{2-[({cyclopropyl[(4-methoxyphenyl)imino]methyl}thio)methyl]phenyl}-3-methoxyacrylate, methyl 1-(2,2-dimethyl-2,3-dihydro-1H-inden-1-yl)-1H-imidazole-5-carboxylate, methyl isothiocyanate, metrafenone, mildiomycin, N-(3′,4′-dichloro-5-fluorobiphenyl-2-yl)-3-(difluoromethyl)-1-methyl-1H-pyrazole-4-carboxamide, N-(3-ethyl-3,5,5-trimethylcyclohexyl)-3-(formylamino)-2-hydroxybenzamide, N-(4-chloro-2-nitrophenyl)-N-ethyl-4-methylbenzenesulfonamide, N-(4-chlorobenzyl)-3-[3-methoxy-4-(prop-2-yn-1-yloxy)phenyl]propanamide, N-[(4-chlorophenyl)(cyano)methyl]-3-[3-methoxy-4-(prop-2-yn-1-yloxy)phenyl]propanamide, N-[(5-bromo-3-chloropyridin-2-yl)methyl]-2,4-dichloronicotinamide, N-[1-(5-bromo-3-chloropyridin-2-yl)ethyl]-2,4-dichloronicotinamide, N-[1-(5-bromo-3-chloropyridin-2-yl)ethyl]-2-fluoro-4-iodonicotinamide, N-[2-(1,3-dimethylbutyl)phenyl]-5-fluoro-1,3-dimethyl-1H-pyrazole-4-carboxamide, N-{(Z)-[(cyclopropylmethoxy)imino][6-(difluoromethoxy)-2,3-difluorophenyl]methyl}-2-phenylacetamide, N-{2-[1,1′-bi(cyclopropyl)-2-yl]phenyl}-3-(difluoromethyl)-1-methyl-1H-pyrazole-4-carboxamide, N-{2-[3-chloro-5-(trifluoromethyl)pyridin-2-yl]ethyl}-2-(trifluoromethyl)benzamide, natamycin, N-ethyl-N-methyl-N′-{2-methyl-5-(trifluoromethyl)-4-[3-(trimethylsilyl)propoxy]phenyl}imidoformamide, N-ethyl-N-methyl-N′-{2-methyl-5-(difluoromethyl)-4-[3-(trimethylsilyl)propoxy]phenyl}imidoformamide, nickel dimethyldithiocarbamate, nitrothal-isopropyl, O-{1-[(4-methoxyphenoxy)methyl]2,2-dimethylpropyl}1H-imidazole-1-carbothioate, octhilinone, oxamocarb, oxyfenthiin, pentachlorophenol and salts, phosphorous acid and its salts, piperalin, propamocarb fosetylate, propanosine-sodium, proquinazid, pyribencarb, pyrrolnitrine, quintozene, S-allyl-5-amino-2-isopropyl-4-(2-methylphenyl)-3-oxo-2,3-dihydro-1H-pyrazole-1-carbothioate, tecloftalam, tecnazene, triazoxide, trichlamide, valiphenal, zarilamid.

The composition according to the invention comprising a mixture of a compound of formula (I) with a bactericide compound may also be particularly advantageous. Examples of suitable bactericide mixing partners may be selected in the following list: bronopol, dichlorophen, nitrapyrin, nickel dimethyldithiocarbamate, kasugamycin, octhilinone, furancarboxylic acid, oxytetracycline, probenazole, streptomycin, tecloftalam, copper sulphate and other copper preparations.

The fungicidal compositions of the present invention can be used to curatively or preventively control the phytopathogenic fungi of crops. Thus, according to a further aspect of the present invention, there is provided a method for curatively or preventively controlling the phytopathogenic fungi of crops characterised in that a fungicidal composition as hereinbefore defined is applied to the seed, the plant and/or to the fruit of the plant or to the soil in which the plant is growing or in which it is desired to grow.

The composition as used against phytopathogenic fungi of crops comprises an effective and non-phytotoxic amount of an active material of general formula (I).

The expression “effective and non-phytotoxic amount” means an amount of composition according to the invention which is sufficient to control or destroy the fungi present or liable to appear on the crops, and which does not entail any appreciable symptom of phytotoxicity for the said crops. Such an amount can vary within a wide range depending on the fungus to be controlled, the type of crop, the climatic conditions and the compounds included in the fungicidal composition according to the invention.

This amount can be determined by systematic field trials, which are within the capabilities of a person skilled in the art.

The method of treatment according to the present invention is useful to treat propagation material such as tubers or rhizomes, but also seeds, seedlings or seedlings pricking out and plants or plants pricking out. This method of treatment can also be useful to treat roots. The method of treatment according to the present invention can also be useful to treat the overground parts of the plant such as trunks, stems or stalks, leaves, flowers and fruits of the concerned plant.

Among the plants that can be protected by the method according to the present invention, mention may be made of cotton; flax; vine; fruit or vegetable crops such as Rosaceae sp. (for instance pip fruit such as apples and pears, but also stone fruit such as apricots, almonds and peaches), Ribesioidae sp., Juglandaceae sp., Betulaceae sp., Anacardiaceae sp., Fagaceae sp., Moraceae sp., Oleaceae sp., Actinidaceae sp., Lauraceae sp., Musaceae sp. (for instance banana trees and plantins), Rubiaceae sp., Theaceae sp., Sterculiceae sp., Rutaceae sp. (for instance lemons, oranges and grapefruit); Solanaceae sp. (for instance tomatoes), Liliaceae sp., Asteraceae sp. (for instance lettuces), Umbelliferae sp., Cruciferae sp., Chenopodiaceae sp., Cucurbitaceae sp., Papilionaceae sp. (for instance peas), Rosaceae sp. (for instance strawberries); major crops such as Graminae sp. (for instance maize, lawn or cereals such as wheat, rice, barley and triticale), Asteraceae sp. (for instance sunflower), Cruciferae sp. (for instance colza), Fabacae sp. (for instance peanuts), Papilionaceae sp. (for instance soybean), Solanaceae sp. (for instance potatoes), Chenopodiaceae sp. (for instance beetroots); horticultural and forest crops; as well as genetically modified homologues of these crops.

Among the diseases of plants or crops that can be controlled by the method according to the present invention, mention may be made of:

Powdery mildew diseases such as:

Blumeria diseases, caused for example by Blumeria graminis;

Podosphaera diseases, caused for example by Podosphaera leucotricha;

Sphaerotheca diseases, caused for example by Sphaerotheca fuliginea;

Uncinula diseases, caused for example by Uncinula necator;

Rust diseases such as:

Gymnosporangium diseases, caused for example by Gymnosporangium sabinae;

Hemileia diseases, caused for example by Hemileia vastatrix;

Phakopsora diseases, caused for example by Phakopsora pachyrhizi or Phakopsora meibomiae;

Puccinia diseases, caused for example by Puccinia recondita;

Uromyces diseases, caused for example by Uromyces appendiculatus;

Oomycete diseases such as:

Bremia diseases, caused for example by Bremia lactucae;

Peronospora diseases, caused for example by Peronospora pisi or P. brassicae;

Phytophthora diseases, caused for example by Phytophthora infestans;

Plasmopara diseases, caused for example by Plasmopara viticola;

Pseudoperonospora diseases, caused for example by Pseudoperonospora humuli or Pseudoperonospora cubensis;

Pythium diseases, caused for example by Pythium ultimum;

Leafspot, leaf blotch and leaf blight diseases such as:

Alternaria diseases, caused for example by Alternaria solani;

Cercospora diseases, caused for example by Cercospora beticola;

Cladiosporum diseases, caused for example by Cladiosporium cucumerinum;

Cochliobolus diseases, caused for example by Cochliobolus sativus;

Colletotrichum diseases, caused for example by Colletotrichum lindemuthanium;

Cycloconium diseases, caused for example by Cycloconium oleaginum;

Diaporthe diseases, caused for example by Diaporthe citri;

Elsinoe diseases, caused for example by Elsinoe fawcettii;

Gloeosporium diseases, caused for example by Gloeosporium laeticolor;

Glomerella diseases, caused for example by Glomerella cingulata;

Guignardia diseases, caused for example by Guignardia bidwelli;

Leptosphaeria diseases, caused for example by Leptosphaeria maculans; Leptosphaeria nodorum;

Magnaporthe diseases, caused for example by Magnaporthe grisea;

Mycosphaerella diseases, caused for example by Mycosphaerella graminicola; Mycosphaerella arachidicola; Mycosphaerella fijiensis;

Phaeosphaeria diseases, caused for example by Phaeosphaeria nodorum;

Pyrenophora diseases, caused for example by Pyrenophora teres;

Ramularia diseases, caused for example by Ramularia collo-cygni;

Rhynchosporium diseases, caused for example by Rhynchosporium secalis;

Septoria diseases, caused for example by Septoria apii or Septoria lycopercisi;

Typhula diseases, caused for example by Typhula incarnata;

Venturia diseases, caused for example by Venturia inaequalis;

Root and stem diseases such as:

Corticium diseases, caused for example by Corticium graminearum;

Fusarium diseases, caused for example by Fusarium oxysporum;

Gaeumannomyces diseases, caused for example by Gaeumannomyces graminis;

Rhizoctonia diseases, caused for example by Rhizoctonia solani;

Tapesia diseases, caused for example by Tapesia acuformis;

Thielaviopsis diseases, caused for example by Thielaviopsis basicola;

Ear and panicle diseases such as:

Alternaria diseases, caused for example by Alternaria spp.;

Aspergillus diseases, caused for example by Aspergillus flavus;

Cladosporium diseases, caused for example by Cladosporium spp.;

Claviceps diseases, caused for example by Claviceps purpurea;

Fusarium diseases, caused for example by Fusarium culmorum;

Gibberella diseases, caused for example by Gibberella zeae;

Monographella diseases, caused for example by Monographella nivalis;

Smut and bunt diseases such as:

Sphacelotheca diseases, caused for example by Sphacelotheca reiliana;

Tilletia diseases, caused for example by Tilletia caries;

Urocystis diseases, caused for example by Urocystis occulta;

Ustilago diseases, caused for example by Ustilago nuda;

Fruit rot and mould diseases such as:

Aspergillus diseases, caused for example by Aspergillus flavus;

Botrytis diseases, caused for example by Botrytis cinerea;

Penicillium diseases, caused for example by Penicillium expansum;

Sclerotinia diseases, caused for example by Sclerotinia sclerotiorum;

Verticilium diseases, caused for example by Verticilium alboatrum;

Seed and soilborne decay, mould, wilt, rot and damping-off diseases:

Alternaria diseases caused for example by Alternaria brassicicola;

Aphanomyces diseases caused for example by Aphanomyces euteiches;

Ascochyta diseases caused for example by Ascochyta lentis;

Aspergillus diseases caused for example by Aspergillus flavus;

Cladosporium diseases caused for example by Cladosporium herbarum;

Cochliobolus diseases caused for example by Cochliobolus sativus

(Conidiaform: Drechslera, Bipolaris Syn: Helminthosporium);

Colletotrichum diseases caused for example by Colletotrichum coccodes;

Fusarium diseases caused for example by Fusarium culmorum;

Gibberella diseases caused for example by Gibberella zeae;

Macrophomina diseases caused for example by Macrophomina phaseolina;

Monographella diseases caused for example by Monographella nivalis;

Penicillium diseases caused for example by Penicillium expansum;

Phoma diseases caused for example by Phoma lingam;

Phomopsis diseases caused for example by Phomopsis sojae;

Phytophthora diseases caused for example by Phytophthora cactorum;

Pyrenophora diseases caused for example by Pyrenophora graminea;

Pyricularia diseases caused for example by Pyricularia oryzae;

Pythium diseases caused for example by Pythium ultimum;

Rhizoctonia diseases caused for example by Rhizoctonia solani;

Rhizopus diseases caused for example by Rhizopus oryzae;

Sclerotium diseases caused for example by Sclerotium rolfsii;

Septoria diseases caused for example by Septoria nodorum;

Typhula diseases caused for example by Typhula incarnata;

Verticillium diseases caused for example by Verticillium dahliae;

Canker, broom and dieback diseases such as:

Nectria diseases, caused for example by Nectria galligena;

Blight diseases such as:

Monilinia diseases, caused for example by Monilinia laxa;

Leaf blister or leaf curl diseases such as:

Taphrina diseases, caused for example by Taphrina deformans;

Decline diseases of wooden plants such as:

Esca diseases, caused for example by Phaemoniella clamydospora;

Diseases of flowers and Seeds such as:

Botrytis diseases, caused for example by Botrytis cinerea;

Diseases of tubers such as:

Rhizoctonia diseases, caused for example by Rhizoctonia solani;

Helminthosporium diseases, caused for example by Helminthosporium solani.

The fungicide composition according to the present invention may also be used against fungal diseases liable to grow on or inside timber. The term “timber” means all types of species of wood, and all types of working of this wood intended for construction, for example solid wood, high-density wood, laminated wood, and plywood. The method for treating timber according to the invention mainly consists in contacting one or more compounds of the present invention, or a composition according to the invention; this includes for example direct application, spraying, dipping, injection or any other suitable means.

The dose of active material usually applied in the treatment according to the present invention is generally and advantageously between 10 and 800 g/ha, preferably between 50 and 300 g/ha for applications in foliar treatment. The dose of active substance applied is generally and advantageously between 2 and 200 g per 100 kg of seed, preferably between 3 and 150 g per 100 kg of seed in the case of seed treatment. It is clearly understood that the doses indicated above are given as illustrative examples of the invention. A person skilled in the art will know how to adapt the application doses according to the nature of the crop to be treated.

The fungicidal composition according to the present invention may also be used in the treatment of genetically modified organisms with the compounds according to the invention or the agrochemical compositions according to the invention. Genetically modified plants are plants into whose genome a heterologous gene encoding a protein of interest has been stably integrated. The expression “heterologous gene encoding a protein of interest” essentially means genes which give the transformed plant new agronomic properties, or genes for improving the agronomic quality of the transformed plant.

The compositions according to the present invention may also be used for the preparation of composition useful to curatively or preventively treat human and animal fungal diseases such as, for example, mycoses, dermatoses, trichophyton diseases and candidiases or diseases caused by Aspergillus spp., for example Aspergillus fumigatus.

The aspects of the present invention will now be illustrated with reference to the following tables of compounds and examples. The following Table illustrates in a non-limiting manner examples of fungicidal compounds according to the present invention. In the following Examples, M+1 (or M−1) means the molecular ion peak, plus or minus 1 a.m.u. (atomic mass units) respectively, as observed in mass spectroscopy and M (ApcI+) means the molecular ion peak as it was found via positive atmospheric pressure chemical ionisation in mass spectroscopy.

TABLE A

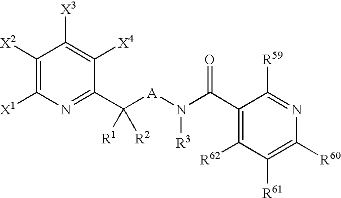

| Compound | A | R¹ | R² | R³ | X¹ | X² | X³ | X⁴ | R⁵⁹ | R⁶⁰ | R⁶¹ | R⁶² | (M + 1) |
|---|---|---|---|---|---|---|---|---|---|---|---|---|---|
| A-1 | O | H | H | H | H | CF3 | H | Cl | Cl | H | H | H | 367 |
| A-2 | O | H | H | H | H | CF3 | H | Cl | SEt | H | H | H | 392 |

TABLE B

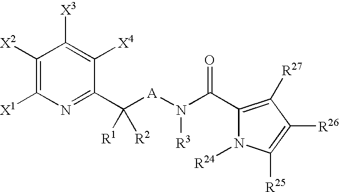

| Compound | A | R¹ | R² | R³ | X¹ | X² | X³ | X⁴ | R²⁴ | R²⁵ | R²⁶ | R²⁷ | (M + 1) |
|---|---|---|---|---|---|---|---|---|---|---|---|---|---|
| B-1 | O | H | H | H | H | CF3 | H | Cl | Me | H | H | H | 334 |

TABLE C

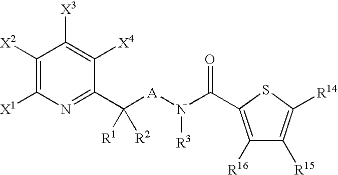

| Compound | A | R¹ | R² | R³ | X¹ | X² | X³ | X⁴ | R¹⁴ | R¹⁵ | R¹⁶ | (M + 1) |
|---|---|---|---|---|---|---|---|---|---|---|---|---|
| C-1 | O | H | H | H | H | CF3 | H | Cl | H | H | Me | 351 |

### EXAMPLES OF PROCESS FOR THE PREPARATION OF THE COMPOUND OF GENERAL FORMULA (I)

### Example 1

### Preparation of N-{[3-chloro-5-(trifluoromethyl)pyridin-2-yl]methoxy}-3-methylthiophene-2-carboxamide (Compound C-1)

150 mg of 2-[(aminooxy)methyl]-3-chloro-5-(trifluoromethyl)pyridine (0.00066 mol), 81 mg (0.00046 mol) of 3-methylthiophene-2-carboxylic acid and 201 mg of 4-(4,6-dimethoxy[1.3.5]triazin-2-yl)-4-methylmorpholinium chloride hydrate (0.00073 mol) are stirred in 2 ml of methanol at room temperature for three hours.

After concentration of the solvent, ethyl acetate is added (5 ml) to the reaction mixture, which is washed with a 1M aqueous solution of hydrogen chloride (5 ml) and then with a saturated aqueous solution of sodium bicarbonate (5 ml).

After separation, the organic phase is dried over magnesium sulfate, filtered, concentrated to dryness and purified on silica to yield to 90 mg of N-{[3-chloro-5-(trifluoromethyl)pyridin-2-yl]methoxy}-3-methylthiophene-2-carboxamide (39%). Mass spectrum: [M+1]=351

### EXAMPLES OF BIOLOGICAL ACTIVITY OF THE COMPOUND OF GENERAL FORMULA (I)

### Example A

### In Vivo Test on Alternaria brassicae (Leaf Spot of Crucifers)

The active ingredients tested are prepared by homogenization in a mixture of acetone/tween/DMSO, then diluted with water to obtain the desired active material concentration.

Radish plants (Pernot variety), sown on a 50/50 peat soil-pozzolana substrate in starter cups and grown at 18-20° C., are treated at the cotyledon stage by spraying with the active ingredient prepared as described above.

Plants, used as controls, are treated with the mixture of acetone/tween/DMSO/water not containing the active material.

After 24 hours, the plants are contaminated by spraying them with an aqueous suspension of Alternaria brassicae spores (40,000 spores per ml). The spores are collected from a 12 to 13 days-old culture.

The contaminated radish plants are incubated for 6-7 days at about 18° C., under a humid atmosphere.

Grading is carried out 6 to 7 days after the contamination, in comparison with the control plants.

Under these conditions, good (at least 70%) or total protection is observed at a dose of 330 ppm with the following compounds: A-2 and C-1.

### Example B

### In Vivo Test on Botrytis cinerea (Grey Mould)

The active ingredients tested are prepared by homogenization in a mixture of acetone/tween/DMSO, then diluted with water to obtain the desired active material Gherkin plants (Vert petit de Paris variety), sown on a 50/50 peat soil-pozzolana substrate in starter cups and grown at 18-20° C., are treated at the cotyledon Z11 stage by spraying with the active ingredient prepared as described above.

Plants, used as controls, are treated with the mixture of acetone/tween/DMSO/water not containing the active material.

After 24 hours, the plants are contaminated by depositing drops of an aqueous suspension of Botrytis cinerea spores (150,000 spores per ml) on upper surface of the leaves. The spores are collected from a 15-day-old culture and are suspended in a nutrient solution composed of:

20 g/L of gelatin

50 g/L of cane sugar

2 g/L of NH4NO3

1 g/L of KH2PO4

The contaminated cucumber plants are settled for 5/7 days in a climatic room at 15-11° C. (day/night) and at 80% relative humidity.

Grading is carried out 5/7 days after the contamination, in comparison with the control plants.

Under these conditions, good (at least 70%) or total protection is observed at a dose of 330 ppm with the following compounds: B-1 and C-1.

### Example C

### In Vivo Test on Pyrenophora teres (Barley Net Blotch)

The active ingredients tested are prepared by homogenization in a mixture of acetone/tween/DMSO, then diluted with water to obtain the desired active material concentration.

Barley plants (Express variety), sown on a 50/50 peat soil-pozzolana substrate in starter cups and grown at 12° C., are treated at the 1-leaf stage (10 cm tall) by spraying with the active ingredient prepared as described above. Plants, used as controls, are treated with the mixture of acetone/tween/DMSO/water not containing the active material.

After 24 hours, the plants are contaminated by spraying them with an aqueous suspension of Pyrenophora teres spores (12,000 spores per ml). The spores are collected from a 12-day-old culture. The contaminated barley plants are incubated for 24 hours at about 20° C. and at 100% relative humidity, and then for 12 days at 80% relative humidity.

Grading is carried out 12 days after the contamination, in comparison with the control plants.

Under these conditions, good (at least 70%) or total protection is observed at a dose of 330 ppm with the following compound: C-1.

### Example D

### Cell Test on Alternaria alternata

The growth of Alternaria alternata is performed on PDA medium at 20° C. under black light during 14 days. The PDA medium is prepared by mixing 39 grams of PDA (Merck) in 1 liter of demineralized water.

The medium is sterilized by autoclave 15 minutes at 121° C. After 14 days of growth, the spores of Alternaria alternata are recovered in sterilized water and the concentration of spores adjusted at 10⁶ spores per ml.

The compounds is solubilized in DMSO and added to sterile liquid glucose/mycopeptone medium (14.6 g/l of D-glucose, 7.1 g/l of mycological peptone (Oxoid) and 1.4 g/l of yeast extract (Merck)) at a concentration of 2 ppm. The medium is inoculated with the spore suspension at a concentration of 10⁵ spores per ml.

The efficacy of the compounds is assessed by OD measurement at 620 nm after 5 days at 20° C. in comparison with a control.

Under these conditions, good (at least 50%) or total protection is observed at the dose of 2 ppm for the following compound: A-1.

Under similar conditions, 2,6-dichloro-N-{[3-chloro-5-(trifluoromethyl)pyridin-2-yl]methoxy}benzamide and N-{[3-chloro-5-(trifluoromethyl)pyridin-2-yl]methoxy}-2,6-difluorobenzamide did not show any activity.

Under similar conditions, 3-hydroxy-4-(1H-imidazol-1-yl)-N-(2-pyridin-2-ylethyl)pyridine-2-carboxamide did not show any activity.

1. A compound of formula (I) 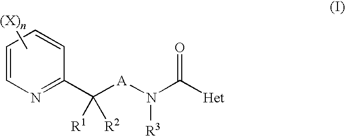
in which:
n is 1, 2, 3 or 4;
X is a halogen atom, a nitro group, a cyano group, a hydroxy group, an amino group, a sulfanyl group, a pentafluoro-λ⁶-sulfanyl group, a formyl group, a formyloxy group, a formylamino group, a carboxy group, a carbamoyl group, a N-hydroxycarbamoyl group, a carbamate group, a (hydroxyimino)-C₁-C₆-alkyl group, a C₁-C₈-alkyl, a C₂-C₈-alkenyl, a C₂-C₈-alkynyl, a tri(C₁-C₈-alkyl)silyl, a C₁-C₈-alkylamino, a di-C₁-C₈-alkylamino, a C₁-C₈-alkoxy, a C₁-C₆-haloalkyl group, a C₁-C₈-halogenoalkoxy having 1 to 5 halogen atoms, a C₁-C₈-alkylsulfanyl, a C₁-C₈-halogenoalkylsulfanyl having 1 to 5 halogen atoms, a C₂-C₈-alkenyloxy, a C₂-C₈-halogenoalkenyloxy having 1 to 5 halogen atoms, a C₃-C₈-alkynyloxy, a C₃-C₈-halogenoalkynyloxy having 1 to 5 halogen atoms, a C₃-C₈-cycloalkyl, a C₃-C₈-halogenocycloalkyl having 1 to 5 halogen atoms, a C₁-C₈-alkylcarbonyl, a C₁-C₈-halogenoalkylcarbonyl having 1 to 5 halogen atoms, a C₁-C₈-alkylcarbamoyl, a di-C₁-C₈-alkylcarbamoyl, a N—C₁-C₈-alkyloxycarbamoyl, a C₁-C₈-alkoxycarbamoyl, a N—C₁-C₈-alkyl-C₁-C₈-alkoxycarbamoyl, a C₁-C₈-alkoxycarbonyl, a C₁-C₈-halogenoalkoxycarbonyl having 1 to 5 halogen atoms, a C₁-C₈-alkylcarbonyloxy, a C₁-C₈-halogenoalkylcarbonyloxy having 1 to 5 halogen atoms, a C₁-C₈-alkylcarbonylamino, a C₁-C₈-halogenoalkylcarbonylamino having 1 to 5 halogen atoms, a C₁-C₈-alkylaminocarbonyloxy, a di-C₁-C₈-alkylaminocarbonyloxy, a C₁-C₈-alkyloxycarbonyloxy, a C₁-C₈-alkylsulphenyl, a C₁-C₈-halogenoalkylsulphenyl having 1 to 5 halogen atoms, a C₁-C₈-alkylsulphinyl, a C₁-C₈-halogenoalkylsulphinyl having 1 to 5 halogen atoms, a C₁-C₈-alkylsulphonyl, a C₁-C₈-halogenoalkylsulphonyl having 1 to 5 halogen atoms, a C₁-C₆-alkoxyimino, a (C₁-C₆-alkoxyimino)-C₁-C₆-alkyl, a (C₁-C₆-alkenyloxyimino)-C₁-C₆-alkyl, a (C₁-C₆-alkynyloxyimino)-C₁-C₆-alkyl, a (benzyloxyimino)-C₁-C₆-alkyl, a benzyloxy, a benzylsulfanyl, a benzylamino, a phenoxy, a phenylsulfanyl or a phenylamino;
A is an oxygen atom, a NR³ group, a sulphur atom, a sulphinyl group, a sulphonyl group or a SiR⁴R⁵ group;
R¹ and R² are chosen independently of each other as being a hydrogen atom, a halogen atom, a cyano group, a hydroxy group, an amino group, a sulfanyl group, a formyl group, a formyloxy group, a formylamino group, a carboxy group, a carbamoyl group, a N-hydroxycarbamoyl group, a carbamate group, a (hydroxyimino)-C₁-C₆-alkyl group, a C₁-C₆-alkyl, a C₂-C₆-alkenyl, a C₂-C₆-alkynyl, a tri(C₁-C₈-alkyl)silyl, a C₁-C₆-alkylamino, a di-C₁-C₆-alkylamino, a C₁-C₆-alkoxy, a C₁-C₆-halogenoalkyl having 1 to 5 halogen atoms, a C₁-C₆-halogenoalkoxy having 1 to 5 halogen atoms, a C₁-C₆-alkylsulfanyl, a C₁-C₆-halogenoalkylsulfanyl having 1 to 5 halogen atoms, a C₂-C₆-alkenyloxy, a C₂-C₆-halogenoalkenyloxy having 1 to 5 halogen atoms, a C₃-C₆-alkynyloxy, a C₃-C₆-halogenoalkynyloxy having 1 to 5 halogen atoms, a C₃-C₆-cycloalkyl, a C₃-C₆-halogenocycloalkyl having 1 to 5 halogen atoms, a C₁-C₆-alkylcarbonyl, a C₁-C₆-halogenoalkylcarbonyl having 1 to 5 halogen atoms, a C₁-C₆-alkylcarbamoyl, a di-C₁-C₆-alkylcarbamoyl, a N—C₁-C₆-alkyloxycarbamoyl, a C₁-C₆-alkoxycarbamoyl, a N—C₁-C₆-alkyl-C₁-C₆-alkoxycarbamoyl, a C₁-C₆-alkoxycarbonyl, a C₁-C₆-halogenoalkoxycarbonyl having 1 to 5 halogen atoms, a C₁-C₆-alkylcarbonyloxy, a C₁-C₆-halogenoalkylcarbonyloxy having 1 to 5 halogen atoms, a C₁-C₆-alkylcarbonylamino, a C₁-C₆-halogenoalkylcarbonylamino having 1 to 5 halogen atoms, a C₁-C₆-alkylaminocarbonyloxy, a di-C₁-C₆-alkylaminocarbonyloxy, a C₁-C₆-alkyloxycarbonyloxy, a C₁-C₆-alkylsulphenyl, a C₁-C₆-halogenoalkylsulphenyl having 1 to 5 halogen atoms, a C₁-C₆-alkylsulphinyl, a C₁-C₆-halogenoalkylsulphinyl having 1 to 5 halogen atoms, a C₁-C₆-alkylsulphonyl, a C₁-C₆-halogenoalkylsulphonyl having 1 to 5 halogen atoms, a benzyl, a benzyloxy, a benzylsulfanyl, a benzylsulfinyl, a benzylsulfonyl, a benzylamino, a phenoxy, a phenylsulfanyl, a phenylsulfinyl, a phenylsulfonyl, a phenylamino, a phenylcarbonylamino or a phenyl group;
with the proviso that R¹ and R² are not both a hydroxyl group;
R³ is a hydrogen atom, a C₁-C₆-alkyl, or a C₃-C₇-cycloalkyl;
R⁴ and R⁵ are chosen independently of each other as being a hydrogen atom, a cyano group, a hydroxy group, a formyl group, a C₁-C₆-alkyl, a C₂-C₆-alkenyl, a C₂-C₆-alkynyl, a tri(C₁-C₈-alkyl)silyl, a C₁-C₆-alkylamino, a di-C₁-C₆-alkylamino, a C₁-C₆-alkoxy, a C₁-C₆-halogenoalkyl having 1 to 5 halogen atoms, a C₁-C₆-halogenoalkoxy having 1 to 5 halogen atoms, a C₂-C₆-alkenyloxy, a C₂-C₆-halogenoalkenyloxy having 1 to 5 halogen atoms, a C₃-C₆-alkynyloxy, a C₃-C₆-halogenoalkynyloxy having 1 to 5 halogen atoms, a C₃-C₆-cycloalkyl, a C₃-C₆-halogenocycloalkyl having 1 to 5 halogen atoms, a C₁-C₆-alkylcarbonyl, a C₁-C₆-halogenoalkylcarbonyl having 1 to 5 halogen atoms, a C₁-C₆-alkylcarbamoyl, a di-C₁-C₆-alkylcarbamoyl, a N—C₁-C₆-alkyloxycarbamoyl, a C₁-C₆-alkoxycarbamoyl, a N—C₁-C₆-alkyl-C₁-C₆-alkoxycarbamoyl, a C₁-C₆-alkoxycarbonyl, a C₁-C₆-halogenoalkoxycarbonyl having 1 to 5 halogen atoms a C₁-C₆-alkylaminocarbonyloxy, a di-C₁-C₆-alkylaminocarbonyloxy, a C₁-C₆-alkyloxycarbonyloxy, a C₁-C₆-alkylsulphenyl, a C₁-C₆-halogenoalkylsulphenyl having 1 to 5 halogen atoms, a C₁-C₆-alkylsulphinyl, a C₁-C₆-halogenoalkylsulphinyl having 1 to 5 halogen atoms, a C₁-C₆-alkylsulphonyl, a C₁-C₆-halogenoalkylsulphonyl having 1 to 5 halogen atoms, a benzyl, a benzylsulfanyl, a benzylsulfinyl, a benzylsulfonyl, a phenylsulfinyl, a phenylsulfonyl, a phenylamino or a phenyl group; and
Het represents a 5-, 6- or 7-membered non-fused heterocycle with one, two or three heteroatoms which may be the same or different, Het being linked by a carbon atom and Het being optionally substituted by one or further substituents chosen independently of each other from a halogen atom, a nitro group, a cyano group, a hydroxy group, an amino group, a sulfanyl group, a pentafluoro-λ⁶-sulfanyl group, a formyl group, a formyloxy group, a formylamino group, a carboxy group, a C₁-C₈-alkyl, a C₁-C₈-halogenoalkyl having 1 to 5 halogen atoms, a C₂-C₈-alkenyl, a C₂-C₈-alkynyl, a C₁-C₈-alkylamino, a di-C₁-C₈-alkylamino, a C₁-C₈-alkoxy, a C₁-C₈-halogenoalkoxy having 1 to 5 halogen atoms, a C₁-C₈-alkoxy-C₂-C₈-alkenyl, a C₁-C₈-alkylsulfanyl, a C₁-C₈-halogenoalkylsulfanyl having 1 to 5 halogen atoms, a C₁-C₈-alkoxycarbonyl, a C₁-C₈-halogenoalkoxycarbonyl having 1 to 5 halogen atoms, a C₁-C₈-alkylcarbonyloxy, a C₁-C₈-halogenoalkylcarbonyloxy having 1 to 5 halogen atoms, a C₁-C₈-alkylsulphenyl, a C₁-C₈-halogenoalkylsulphenyl having 1 to 5 halogen atoms, a C₁-C₈-alkylsulphinyl, a C₁-C₈-halogenoalkylsulphinyl having 1 to 5 halogen atoms, a C₁-C₈-alkylsulphonyl and a C₁-C₈-halogenoalkylsulphonyl having 1 to 5 halogen atoms or a C₁-C₈-alkylsulfonamide;
as well as a salt, an N-oxyde, a metallic complex, a metalloidic complex and/or an optically active isomer thereof;

2. A compound according to claim 1, wherein n is 1 or 2.

3. A compound according to claim 1, wherein X is chosen as being a halogen atom, a cyano group, a (hydroxyimino)-C₁-C₆-alkyl group, a C₁-C₈-alkyl, a C₂-C₈-alkenyl, a C₂-C₈-alkynyl, a tri(C₁-C₈-alkyl)silyl, a C₁-C₈-alkoxy, a C₁-C₆-haloalkyl group, a C₁-C₈-halogenoalkoxy having 1 to 5 halogen atoms, a C₃-C₈-cycloalkyl or a C₃-C₈-halogenocycloalkyl having 1 to 5 halogen atoms.

4. A compound according to claim 1, wherein R¹ and R² are chosen independently of each other as being a hydrogen atom, a halogen atom, a C₁-C₈-alkyl, a C₂-C₈-alkenyl, a C₂-C₈-alkynyl, a tri(C₁-C₈-alkyl)silyl, a C₁-C₈-alkoxy, a C₁-C₆-haloalkyl group, a C₁-C₈-halogenoalkoxy having 1 to 5 halogen atoms, a C₃-C₈-cycloalkyl, or a C₃-C₈-halogenocycloalkyl having 1 to 5 halogen atoms.

5. A compound according to claim 1, wherein A is chosen as being an oxygen atom, a nitrogen atom or a sulphur atom.

6. A compound according to claim 1, wherein R⁴ and R⁵ are chosen independently of each other as being a hydrogen atom, a C₁-C₈-alkyl, a C₂-C₈-alkenyl, a C₂-C₈-alkynyl, a C₁-C₈-alkoxy, a C₁-C₆-haloalkyl group, a C₁-C₈-halogenoalkoxy having 1 to 5 halogen atoms, a C₃-C₈-cycloalkyl or a C₃-C₈-halogenocycloalkyl having 1 to 5 halogen atoms.

7. A compound according to claim 1 wherein p is 1 or 2.

8. A compound according to claim 1, wherein Y is chosen as being a halogen atom, a C₁-C₈-alkyl, a C₁-C₈-alkoxy, a C₁-C₆-haloalkyl group or a C₁-C₈-halogenoalkoxy having 1 to 5 halogen atoms.

9. A compound according to claim 1, wherein Het is chosen as being 2-furan, 3-furan, 4,5-dihydro-3-furan, 2-thiophene, 3-thiophene, 2-pyrrole, 3-pyrrole, 5-oxazole, 4-oxazole, 5-thiazole, 4-thiazole, 5-pyrazole, 4-pyrazole, 3-pyrazole, 3-isoxazole, 4-isoxazole, 5-isoxazole, 3-isothiazole, 4-1,2,3-triazole, 4-thiadiazole, 5-thiadiazole, 2-pyridine, 3-pyridine, 4-pyridine, 2-oxathiine, 4,5-dihydro-3-pyran, 4,5-dihydro-2-thiopyran, 4,5-dihydro-3-thiopyran or 2-pyrazine.

10. A compound according to any of the claim 1, wherein Het is substituted in ortho position.

11. A compound according to claim 1, wherein Het is substituted by a halogen atom, a C₁-C₆-alkyl, an amino group, a C₁-C₈-alkylamino or a di-C₁-C₈-alkylamino.

12. A compound according to claim 1, wherein Het is a five membered ring heterocycle.

13. A compound according to claim 1, wherein Het is a five membered ring heterocycle.

14. A process for the preparation of a compound of formula (I) as defined in claim 1, which comprises reacting a pyridin-2-ylmethyl derivative of formula (II) or a salt thereof: 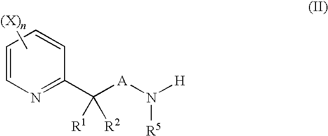
with a carboxylic acid derivative of the formula (III) 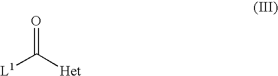
in which:
L¹ is a leaving group chosen as being a halogen atom, a hydroxyl group, —OR^{a}, —OCOR^{a}, R^{a} being a C₁-C₆ alkyl, a C₁-C₆ haloalkyl, a benzyl, 4-methoxybenzyl, pentafluorophenyl or a group of formula 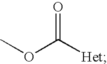
in the presence of a catalyst and, if L¹ is a hydroxyl group, in the presence of a condensing agent.

15. A fungicide composition comprising an effective amount of a compound according to claim 1 and an agriculturally acceptable support.

16. A method for preventively or curatively combating the phytopathogenic fungi of crops, comprising applying an effective and non-phytotoxic amount of a composition according to claim 14 to the plant seeds or to the plant leaves and/or to the fruits of the plants or to the soil in which the plants are growing or in which a plant is desired to grow.

17. Seed treated with a composition of claim 15.

18. A plant grown in contact with a composition of claim 15.

19. A compound according to claim 2, wherein Het is a five membered ring heterocycle.

20. A compound according to claim 3, wherein Het is a five membered ring heterocycle.
